(19) United States
(12) Patent Application Publication
(10) Pub. No.: US 2002/0123074 A1
(43) Pub. Date: Sep. 5, 2002

(54) Method and apparatus for determination of gastrointestinal intolerance

(76) Inventors: Thomas W. Self, La Jolla, CA (US); Linda Self, La Jolla, CA (US)

Correspondence Address: BROWN, MARTIN, HALLER & MCCLAIN LLP, 1660 UNION STREET, SAN DIEGO, CA, 92101-2926, US

(21) Appl. No.: 10/090,856

(22) Filed: Mar. 1, 2002

Related U.S. Application Data
(60) Provisional application No. 60/273,006, filed on Mar. 2, 2001.

(51) Int. Cl. G01N 33/53; G06F 19/00; G01N 33/48; G01N 33/50; C12M 1/34

(52) U.S. Cl. 435/7.1; 435/287.2; 702/19

(57) ABSTRACT

The invention is a standardized stool-based assay kit for the diagnosis of food allergies. The assay is highly reliable and can be performed with no harm to the patient. The assay can be configured in a number of formats for optimization for use in a primary care or large clinical setting.

### CROSS-REFERENCES TO RELATED APPLICATIONS

This application claims the benefit of priority of U.S. provisional application Serial No. 60/273,006 filed Mar. 2, 2001 which is incorporated herein by reference in its entirety.

### BACKGROUND OF THE INVENTION

Adverse reactions to food may be caused by a number of factors, either due to the nature of the food or the individual consuming the food. Toxic reactions are not related to individual sensitivity but instead are due to the nature of the food. Examples of toxic reactions include food poisoning due to bacterial contamination of food or reactions to histamine in scromboid fish. Non-toxic food reactions are due to the individual consuming the food. Reactions may be due to decreased ability to digest food due to decreased digestive enzyme production (e.g. lactose intolerance) or autoimmune disorder (e.g. wheat intolerance in celiac disease). Such reactions constitute food intolerance, as opposed to food allergy, as the body does not produce antibodies to the antigens present in a specific food.

The term “food allergy” refers to adverse immunologic reactions to food. Food allergy is usually mediated by IgE type antibodies directed to specific food proteins, but other immunologic mechanisms can play a role. Reactions may be acute (e.g. anaphylaxis, hives) or chronic (e.g. asthma, atopic dermatitis and gastrointestinal disorders). Due to the potential severity of the reaction, treatment typically consists of elimination of the potential allergen from the diet. This can be problematic and cumbersome if the individual is believed to be allergic to a common food. The most common food allergies in children are milk, egg, peanut, soy, wheat, tree nuts, fish and shellfish. The most common food allergies in adults are peanuts, tree nuts, shellfish and fish (Sicherer, 1999). As reactions to foods may be severe in some individuals, it would be beneficial to be able to determine food allergies without having to expose the individual to potential allergens or re-expose the individual to suspected allergens to test for a response.

Prick-puncture skin tests are the cornerstone of allergy diagnosis for inhaled or contact allergens. Skin testing is cheap, safe and easy to do, highly predictive and may be readily performed in the office of a general practitioner. While the individual is not taking antihistamines, a device such as a bifurcated needle or a lancet is used to puncture the skin through a glycerinated extract of a potential allergen and also through appropriate positive (histamine) and negative (saline-glycerine) controls. A local wheal-and-flare response indicates the presence of IgE antibodies to the specific allergen, with a wheal diameter of more than 3 mm indicating a positive response.

Prick tests for food allergies are most valuable when they are negative because the negative predictive value of these tests is very high (over 95 percent). Unfortunately, the positive predictive value of the tests is on the order of only 50 percent (Bock et al., 1978; Sampson and Albergo, 1984). Thus, a positive skin test in isolation cannot be considered proof of clinically relevant hypersensitivity, whereas a negative test virtually rules out IgE-mediated food allergy to the food in question. Intradermal allergy skin tests with food extracts, a test similar to the prick tests, give an unacceptably high false-positive rate and therefore should not be used.

An in vitro method for testing for food allergies, known as radioallergosorbent tests (RAST) has also been developed. The method is practical for use in a primary care setting, but has similar drawbacks to the skin prick test in terms of levels of predictability. A negative result is highly reliable, but a positive result has a low positive predictive value. The tests are generally less sensitive, but more quantitative than skin tests.

Double-blind, placebo controlled food challenges are considered the gold standard for diagnosing food allergies. The procedure is labor intensive and expensive. Samples must be prepared such that the individual administering the test and the patient are unaware of what foods are contained in each test sample. Testing is ideally carried out in a setting with immediate access to emergency medications including epinephrine, antihistamines, steroids and inhaled beta-agonists, as well as emergency resuscitation equipment, as reactions can be severe and life threatening (Sampson and Albergo 1984). Patients avoid the suspected food for at least two weeks, antihistamine therapy is discontinued for a sufficient time to allow clearance of the drug from the individual depending on the pharmacokinetics of the drug, and asthma medications are reduced to the lowest level possible. Either a challenge food or a placebo is administered hidden in another food or in an opaque capsule.

Patients are observed for reaction, both immediate and delayed. Due to delayed reactions, ideally only one or two potential allergens can be tested per day. If allergy to only a few foods is suspected, single blind or open challenges may be used. Negative challenges are confirmed with open-feeding of a larger, meal-sized portion of the food. Oral challenges should not usually be performed on individuals with a clear history of reactivity.

Food intolerance is common in infants and children under the age of three. In severe cases, malabsorption may result in a syndrome known as failure to thrive. Malabsorption may result from the lack of a digestive enzyme such that nutrients are not obtained from the food consumed. Tests exist that allow for the detection of fats (steatorrhea) and simple sugars in the stool, demonstrating incomplete digestion. In the event that such tests are negative, a number of possible reasons remain for malabsorption. Conversely, parental beliefs about factitious food allergies can result in parents withholding specific foods resulting in failure to thrive (Roesler et al., 1994). A cohort of children believed by their parents to have multiple food allergies, who demonstrated a failure to thrive were tested for food allergies. Four of the 11 children had positive reactions using a prick-puncture skin test. Upon testing children using double-blind, placebo-controlled food challenges, only two of the 11 children reacted to either one or two of the foods presented, respectively. The study reaffirms problems with diagnosis of allergies using the prick-puncture skin test and highlights the need for a method to accurately determine an absence of allergies as well. Moreover, even if skin prick tests were more predictive, subjecting an infant to such a test would be traumatic. Food challenge assays are exceptionally problematic as many infants are breast fed, and many food antigens are able to pass from the mother through the breast milk providing no indication as to the cause of the allergic reaction (e.g. casein, soy).

A method for testing for gastrointestinal protein allergy was described by Self et al. in which stool samples were tested for the presence of anti-food antibodies (Self et al., 1969, incorporated herein by reference). In the study, stool samples from 25 children with chronic diarrhea without determined organic cause were tested for the presence of precipitating antibodies to milk and wheat protein using an agar micro-double immunodiffusion assay. To perform the assay, agarose was melted in an appropriate buffer, placed on a glass slide and allowed to solidify. A series of wells were made individually in the agarose, one for the test sample from the patient in the center surrounded by multiple wells for potential food antigens. The test antigens were whole milk, skim milk, and 5% and 10% solution of barley, wheat, oats, rye, soy and selected purified proteins from various foods. Slides were kept at room temperature in a moist chamber and were examined at 6, 24 and 48 hours. Precipitates were observed qualitatively by placing the plates on a black, back-lit background and examining the plates by eye using a magnification lamp.

All of the children who fulfilled the clinical criteria for the diagnosis of gastrointestinal allergy to milk were tested for the presence of circulating antibodies to milk and found to be negative. This suggested that the immunologic phenomenon might be entirely localized to the gastrointestinal tract. Further studies of the stools of such patients demonstrated the actual presence of precipitating substances or “coproantibodies” to milk. No precipitating antibodies were found in any of the stools from an age-matched healthy control group. A recurrence of diarrhea then developed in two of the original patients while they were taking a milk-protein-free diet and their stools were found to contain precipitating antibodies against wheat proteins.

These findings indicate that the predictive value of a test based on stool is significantly higher than one based on serum. Moreover, the test is completely non-invasive, as opposed to the skin prick test, and presents no danger to the individual, as opposed to a food challenge test. Diagnosis of food allergies would benefit from the availability of such a test. However, the double-micro immunodiffusion assay taught by Self is a non-trivial assay as detailed above. Although the test was found to be highly predictive for food allergies in a research laboratory setting, the test could not be readily performed in a physician's office or in a hospital clinical lab due to the complexities of the assay. The use of unreliable serum based assays persist. No assay based on analysis of stool samples are widely available for testing patients for food allergies in a clinical setting.

### SUMMARY OF THE INVENTION

The invention is a standardized stool-based assay kit for the detection of anti-food antibodies for the diagnosis of food allergies using the methods and test apparatuses of the invention. The assay can be performed in a clinical laboratory or physician's office setting and may exist in a number of formats well known to those skilled in the art including, but not limited to, radial immunodiffusion, agar immunodiffusion, latex agglutination or dipstick assay. Antigens from food allergens, typically common food antigens (e.g. milk, eggs, peanuts, tree nuts) are attached to an appropriate matrix. For example, antigens can be immobilized in or loaded into wells of a gel (e.g. agarose, acrylamide) or applied to a particulate matrix (e.g. latex beads). Stool samples are prepared by separating the solid and liquid components by any of a number of methods including, but not limited to centrifugation or sedimentation over time. The stool supernatant from the potentially allergic individual is applied to at least one discrete region of the matrix to allow for the interaction of the antigen with the antibody. The reaction of the antibody with the antigen is monitored for the formation of a precipitate, preceptin band or aggregate. Aggregates can be visualized directly in the latex agglutination and dipstick assays. Precipitates produced in radial immunodiffusion and agar immunodiffusion assays may be observed directly over time by eye or by the use of an automated system. Precipitate formation results in increased opacity of the gel. Alternatively the gel can be stained with coomassie brilliant blue or other appropriate at a single time end point after washing to remove non-aggregated protein to visualize proteins. The standardized assay kit allows for the comparison of results between individuals and in a single individual over time. Inclusion of the test apparatuses into kits makes them available for widespread use in any of a number of clinical settings. The readouts of the assays are similar to those in a number of assays routinely performed in clinical laboratories.

The test apparatuses of the invention can be configured in a number of formats to allow for the testing of different combinations of antigens and samples. In the most simplified version, a single antigen is tested for interaction with a single sample. In a more complex assay, a single antigen may be tested for interaction with a number of samples, including positive and negative control samples, which may be preferred in larger hospital laboratories in which multiple samples need to be tested at once. Conversely, multiple antigens may be tested for interaction with a single sample, which may be preferred in a primary care setting. The test apparatus can be configured for qualitative analysis for the determination of the presence of anti-food antibodies or semi-quantitatively to monitor the persistence of anti-food antibodies in children. Regardless of the specific configuration of the test apparatus, the antigens are preferably stabilized to prevent loss of antigenicity. The antigens may be whole proteins (e.g. casein, ovalbumin), peptides derived therefrom or mixtures of proteins derived from potentially antigenic foods. Assays using the test apparatuses of the invention can be performed with minimal preparation of the sample and the apparatus for qualitative assays. More substantial preparation of the sample (e.g. preparation of a series of dilutions of the sample and controls) or monitoring of the progress of the assay (e.g. observation of time course of reaction) may be required for quantitative and semi-quantitative assays.

The invention is a machine that performs the assay of the invention using the test apparatus of the invention. The machine contains a centrifuge for the separation of the stool sample, a transfer apparatus to apply a portion of the supernatant to the antigen, an optional environmental chamber to allow for the formation of the precipitate, a reader to detect the formation of a precipitate and a recorder to record data obtained by the reader. It is possible to include a mechanism for washing and staining gels in an immunodiffusion assay before the reading.

### BRIEF DESCRIPTION OF THE DRAWINGS

FIG. 1. Latex agglutination test for interaction of a single sample with a single antigen.

FIG. 2. Gel immunodiffusion assay for multiple samples, top view

FIG. 3. Cross-sectional view of FIG. 2 at (3-3).

FIG. 4. Gel immunodiffusion assay for any combination of antigens and samples, top view.

FIG. 5. Cross-sectional view of FIG. 4 at (5-5).

FIG. 6. Radial immunodiffusion assay apparatus for multiple samples with a single antigen, top view.

FIG. 7. Cross-sectional view of FIG. 6 (7-7).

FIG. 8. Flow chart for steps to be performed using an automated system.

The present invention will be better understood from the following detailed description of preferred embodiments of the invention, taken in conjunction with the accompanying drawings.

### DETAILED DESCRIPTION AND PREFERRED EMBODIMENTS

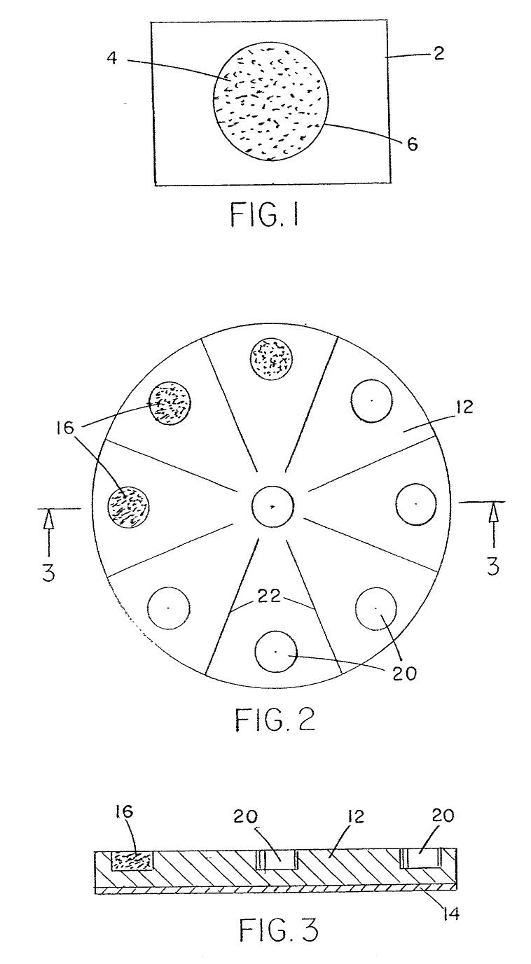

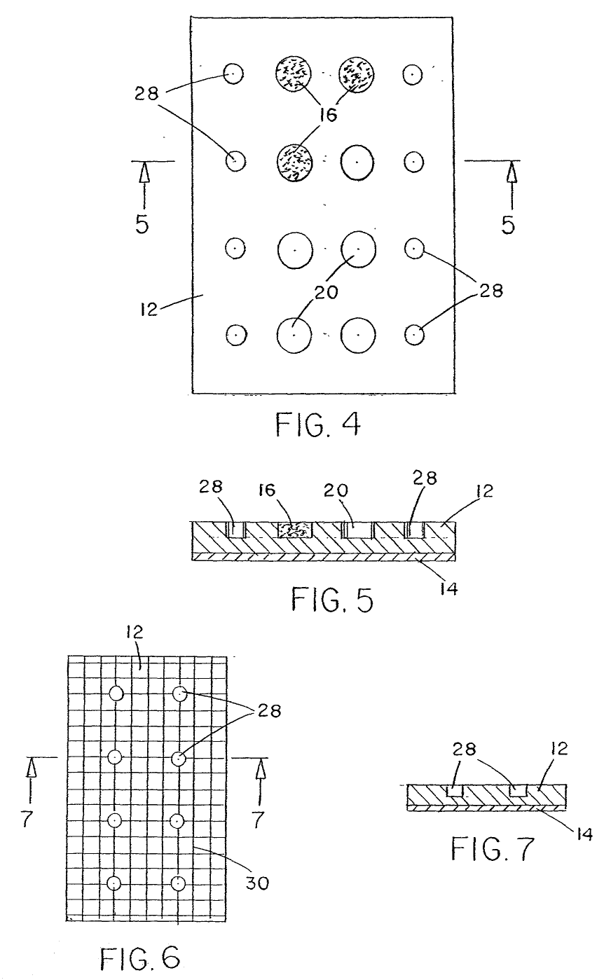

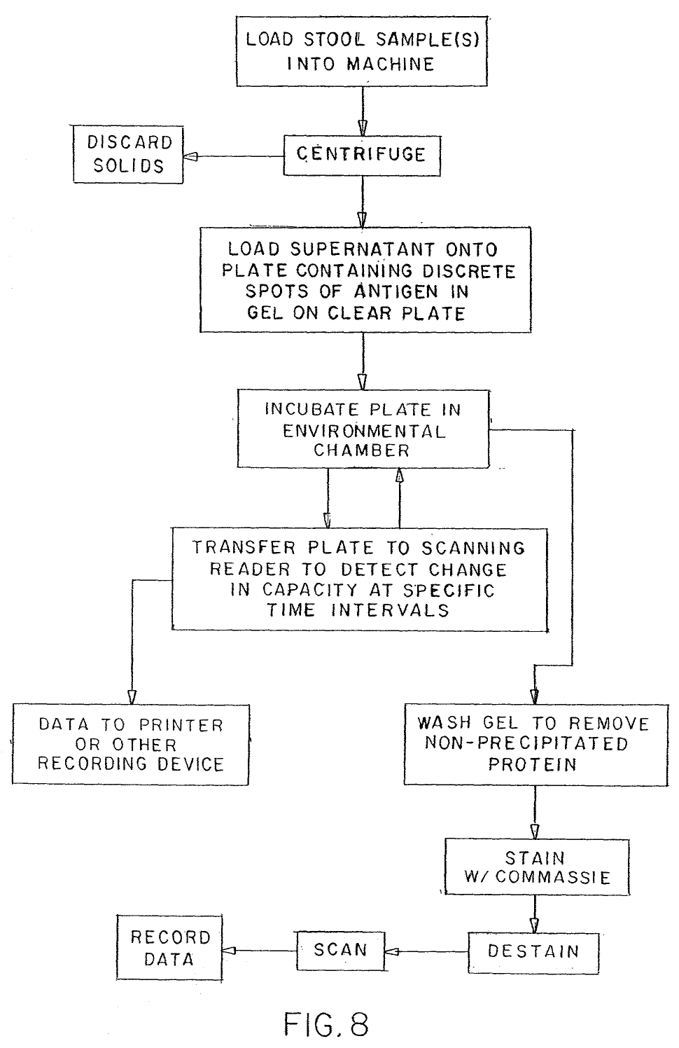

The invention is a standardized stool-based assay kit for the detection of anti-food antibodies for the diagnosis of food allergies using the methods and test apparatuses of the invention. The invention allows for the performance of a highly reliable, non-invasive test that presents no potential harm to the patient. The apparatus can be configured to test for the reaction of one (FIG. 1) or a plurality of samples with a single antigen. Alternatively, the apparatus can be configured to test a single sample for the presence of antibodies to multiple antigens (FIGS. 2-3). A third configuration can be used for any combination of antigens and samples (FIGS. 4-5). In a fourth configuration multiple samples can tested for interaction with a single antigen (FIGS. 6-7). Using such a configuration, a quantitative test can be performed by using a series of dilutions of samples and control reagents. The test apparatuses of the invention can be configured for performance of the test, individually, by hand, in the setting of a physician's office, or in a large scale clinical laboratory using the machine of the invention (FIG. 8).

The test apparatuses and methods of the invention are distinguished from other currently available clinical food allergy tests in that it is designed for use with stool samples to determine the presence of antibodies in the gastrointestinal (GI) tract rather than in the circulation. Serum is tested in RAST, ELISA and western blot assays. Prick-puncture tests also assay for the presence of circulating antibodies. The source of the test sample is an innate limitation of these serum-based assays. Food allergies are due to an allergic reaction in the GI tract, not in circulation. In one report, the predictive accuracy of the methods were compared to each other and double blind placebo food challenge (Sampson and Albergo, 1984). Although both were found to have a high negative predictive value (82-100%), the positive predictive value of the tests was poor and quite variable, 25-75% for prick-puncture tests and 0-57% for RAST. Positive predictive accuracy was not increased by combining the data derived from the two test methods. As such tests are highly predictive for the presence contact or inhaled allergens, their low reliability in detecting anti-food antibodies is not due innately to the test, but instead to the substance tested. Analysis of a bodily substance from the GI tract, i.e. stool, has been shown to have a substantially higher predictive value as the allergic reaction occurs in the GI tract.

The standardized assay kit of the invention allows for the determination of the presence of antibodies without knowledge of the type (e.g. IgA, IgG, IgE, IgM) of antibody present. The test apparatuses and methods of the invention allow for the detection of interaction between an antibody and the antigen. It does not employ a secondary antibody to detect the presence of the antibody from the subject (e.g. in ELISA and RAST). Reaction products are detected by observing the aggregate or precipitate directly or by the use of a non-specific stain (e.g. coomassie brilliant blue). Therefore, a false negative cannot be obtained by using an inappropriate specific reagent.

A number of configurations of potential test apparatuses are detailed in the application. The selection of a single or multiple sample or antigen test is a matter of choice depending on the a number of factors known to those skilled in the art. In a hospital setting or an allergy clinic, it may be advantageous to test the interaction of multiple samples with a single antigen in a single test apparatus. This facilitates the performance of both positive and negative controls using samples from known allergic or non-allergic individuals. A family practitioner who is unlikely to have multiple samples to test at a single time may find it advantageous to have to prepare only a single sample for application to a test apparatus that contains multiple antigen. In a situation where confirmation of a single allergy is required, a test containing a single antigen may be preferable. As allergies present in infancy and early childhood may disappear with age, a quantitative or semi-quantitative assay may be selected to monitor the persistence of allergies in a child.

In a preferred embodiment the assay kits are manufactured and packaged under conditions comparable to other clinical testing kit. These manufacturing standards are well known to those skilled in the art.

### EXAMPLE 1

Test for Reaction to a Single Food Antigen Using Latex Agglutination Assay.

Methods for preparation of protein coated latex beads for use in latex agglutination assays are well known. Briefly, a 3% dyed latex suspension is coated and blocked with an antigen of a potentially allergenic food (e.g. milk, eggs, soy) or a negative control antigen (e.g. lamb, potato, which are typically non-allergenic) as described by Cummins et al. (1994). Optimal concentration of antigen can be determined by agglutination of the coated latex particles with positive and negative control sera. Such optimizations are well within the ability of those skilled in the art. Test and control latex are stabilized by freeze-drying. Latex prepared in this manner is typically stable for at least 12 months at room temperature and demonstrate no loss of activity. After reconstitution, the mixture must be stored at 4° C. Stability varies depending on the antigen, but the latex may be stable for up to 2 months. Ideally, both positive and negative controls should be run at all times. Controls are essential if there is any question regarding the quality of the latex.

The latex agglutination assay is performed by placing 5 μl drops of the supernatant of a stool sample on a white agglutination card. Subsequently, the supernatant drops are mixed with equal volumes of control and test latex suspension. Latex is mixed with the sample using a disposable pipette tip. The card is shaken gently for two minutes and agglutination (clumping of latex) is observed directly. Such analysis can be readily performed by one skilled in the art. The assay can be scored in a semi-quantitative manner using a timer, with more rapidly forming aggregates (those formed in less than 30 seconds) indicating a strong reaction and more slowly forming aggregates (those formed after more than 90 seconds) indicating a weak reaction.

Alternatively, latex can be dried onto individually wrapped agglutination cards (2). The assay is performed by suspending the dried detection reagent (4) quickly and completely in the stool supernatant within a defined area (6) on the card (2) using a spatula or other appropriate tool while the card is maintained on a flat, horizontal surface. The card is rotated laterally on the flat surface to promote agglutination. Results are read and scored promptly as above.

### EXAMPLE 2

Test for Reaction to Multiple Foods in an Individual Sample Using Gel Diffusion.

A stool sample is subject to centrifugation to separate the solids from the supernatant. An individually wrapped test apparatus (FIGS. 2, 3) is removed from the air tight container in which the test apparatus is stored to prevent the gel from drying out. The sample is loaded into the center well (10) of a round immunodiffusion assay gel (12), mounted on a clear support (14), preferably plastic, containing multiple food antigens (e.g. whole proteins, peptides, protein extracts) can be incorporated into discrete regions (16) of the gel (e.g. agarose, acrylamide) during manufacture. Preservatives (e.g. EDTA, sodium azide) and/or protease inhibitors that do not interfere with the reaction (e.g. non-peptide inhibitors) are optionally added to the gel or protein to increase protein stability. Alternatively, a selection of food antigens, preferably freeze dried or stabilized by some other method, can be prepared by resuspension or other method and loaded into antigen wells (20) in the test apparatus. The test apparatus may include barriers (22) to prevent cross-contamination of antigens. The gel is transferred into a humid chamber and incubated at room temperature. After various time points, preferably 6, 24 and 48 hours, the gel is observed for the presence of a preceptin band by eye or using an automated system. After 48 hours, the gel is washed and stained using methods well known to those skilled in the art. Typically the gel is washed in an isotonic solution with multiple (3 to 8) changes of wash solution. The gel is stained using coomassie brilliant blue. Stain not bound to protein is removed by multiple washes in destain. The gel can be dried, photographed or imaged by any of a number of methods for a permanent record of the test.

One can readily design a test apparatus based on this description in which the antigen is loaded into a central well and the samples are loaded into wells in the gel located around the periphery of the test apparatus. The assay is performed as described in the previous paragraph.

### EXAMPLE 3

Test Apparatus for Analysis of any Combination of Antigens and Samples.

A rectangular immunodiffusion assay gel (FIGS. 4-5), mounted on a clear support (14), containing multiple food antigens (e.g. whole proteins, peptides, protein extracts) can be incorporated into discrete regions (16) of the gel (e.g. agarose, acrylamide) during manufacture. Preservatives (e.g. EDTA, sodium azide) and/or protease inhibitors that do not interfere with the reaction (e.g. non-peptide inhibitors) are optionally be added to the gel or protein to increase protein stability. Alternatively, a selection of food antigens, preferably freeze dried or stabilized by some other method, can be prepared and loaded into antigen wells (20) in the test apparatus. The test apparatus may include barriers to prevent cross-contamination of antigens. A number of sample wells (28) equal to the number of discrete antigen regions (16) or wells (20) are present. The supernatant from a single or various stool samples are loaded into the sample wells (28). The assay is performed as in Example 3.

### EXAMPLE 4

Radial Immunodiffusion Rest to Determine Persistence of Anti-Food Antibodies.

A stool sample from a child known to previously have anti-casein antibodies is placed at 4° C. overnight to allow the solids to settle to the bottom of the container. An individually wrapped test apparatus (FIGS. 6-7) is removed from its airtight container in which it is stored to prevent the gel from drying out. The gel (12) is mounted on a clear, solid support (14), optionally with a grid pattern (30) etched into the support (14). The gel (12) contains a known concentration of casein distributed homogeneously throughout the gel. Preservatives and protease inhibitors are optionally added into the gel to increase protein stability. The gel contains a series of sample wells (28) into which a dilution series of the stool supernatant and, ideally, an anti-casein antibody of known concentration, are loaded. The gel is incubated at room temperature in a humid chamber for a defined period of time (typically 6 to 48 hours) to allow for the formation of a perceptin ring. The gel can be observed directly as described above using the grid etched in the solid support to compare the sizes of the rings. Alternatively, the gel can be washed and stained as described above. As perceptin rings are typically not perfectly round, care should be taken in determining the area of the rings. The size of the rings in the test sample are compared to the size of the rings generated by the known antibody. The concentration of the antibodies from test to test can be determined in an individual before attempts to introduce the offending food back into the diet. If no perceptin ring can be detected, the food may be reintroduced into the diet. If the size of the perceptin ring decreases over time, the test should be repeated until no ring is formed before reintroduction of the food.

### EXAMPLE 5

Machine for Automated Analysis of Stool Samples for the Presence of Antibodies.

A machine can be designed for the processing of test apparatuses such as those described in Examples 2-4. The steps are detailed in a flow chart in (FIG. 8). A number of machines are available for the automation of biological assays. Components such as centrifuges, fluid handling devices, environmental chambers, optical density scanners and recording devices, as well as methods for linking such components, are well known to those skilled in the art. In a preferred embodiment of the invention, a stool sample is placed into a tube and the tube is placed into the machine. A test apparatus, such as those described in the previous Examples, is loaded into the machine. The selection of the apparatus is a matter of choice depending on the information desired by the individual requesting the analysis. The machine is programmable to accommodate a number of test apparatuses. The supernatant is separated from the solids of the sample or samples. Fluid handling devices load the supernatant, and possibly the desired protein samples, into the test apparatus. The apparatus is incubated in a humid, environmental chamber for defined time periods which may vary with the assay being performed. The gel is scanned for the formation of a precipitate by scanning the gel for changes in opacity. As an automated scanner is more sensitive than the eye, the use of a scanner may allow for decreased incubation time and/or miniaturization of the test apparatus. The machine may scan the test apparatus repeatedly until a threshold opacity or final time point is reached. The changes in opacity are recorded in computer memory or printed numerically or using a chart recorder. Ideally, the test apparatus contains both negative and positive control regions to set maximum and minimum opacity levels to set ranges on the machine. The machine optionally contains a series of conduits for the passage of wash, stain, destain and waste solutions to stain the gel for final analysis, documentation and storage.

Although an exemplary embodiment of the invention has been described above by way of example only, it will be understood by those skilled in the field that modifications may be made to the disclosed embodiment without departing from the scope of the invention, which is defined by the appended claims.

### REFERENCES

Bock, S A et al. Appraisal of skin tests with food extracts for diagnosis of food hypersensitivity. Clin Allergy 1978; 8:559-64.

Cummins, A J et al. Development of a rapid latex agglutination test for the detection of visceral leishmaniasis. Trans. R. Soc. Trop. Med. Hyg. 88:300.

Sampson, H A & Albergo R. Comparison of results of skin tests, RAST, and double-blind, placebo-controlled food challenges in children with atopic dermatitis. J Allergy Clin Immunol 1984;74:26-33.

Sicherer, S H Manifestations of food allergy: Evaluation and Management. Am. Fam. Physician 1999; 59:415-24

Self, T W et al. Gastrointestinal protein allergy. Immunologic considerations. JAMA 1969; 207:2393-6.

I claim:

1. A standardized assay kit for detection of an anti-food antibody in a stool sample wherein the antibody is detected by observation of a reaction of the antibody with an antigen present in the kit.

2. The standardized assay kit of claim 1, wherein the kit comprises a single antigen for detection of a single antibody in a single stool sample.

3. The standardized assay kit of claim 1, wherein the kit comprises a plurality of antigens for detection of a plurality of antibodies in a single stool sample.

4. The standardized assay kit of claim 1, wherein the kit comprises a single antigen for detection of antibody in a plurality stool samples.

5. The standardized assay kit of claim 1, wherein the kit comprises a plurality of antigens for detection of a plurality of antibodies in a plurality of stool samples.

6. The standardized assay kit of claim 1, wherein the reaction is detected by observation of a precipitate.

7. The standardized assay kit of claim 1, wherein the reaction is detected by observation of an aggregate.

8. The standardized assay kit of claim 1, wherein the assay is performed by hand.

9. The standardized assay kit of claim 1, wherein the assay is performed by an automated system.

10. A standardized assay kit for detection of an anti-food antibody in a stool sample wherein the kit comprises a solid support,
a matrix,
at least one antigen in contact with said matrix,
at least one discrete region on the matrix for application of the stool sample to allow for interaction between the antigen and the sample, wherein the antibody is detected by observation of a reaction of the antibody with an antigen present in the kit.

11. The standardized assay kit of claim 10, wherein the kit comprises a single antigen for detection of a single antibody in a single stool sample.

12. The standardized assay kit of claim 10, wherein the kit comprises a plurality of antigens for detection of a plurality of antibodies in a single stool sample.

13. The standardized assay kit of claim 10, wherein the kit comprises a single antigen for detection of antibody in a plurality stool samples.

14. The standardized assay kit of claim 10, wherein the kit comprises a plurality of antigens for detection of a plurality of antibodies in a plurality of stool samples.

15. The kit of claim 10, wherein the matrix comprises a gel.

16. The kit of claim 15, wherein the antigen is incorporated into the gel.

17. The kit of claim 15, wherein the antigen is loaded into a well in the gel.

18. The kit of claim 10, wherein the matrix comprises latex.

19. The kit of claim 10, wherein the stool sample is placed directly in contact with the antigen.

20. The kit of claim 10, wherein the stool sample is placed adjacent to the antigen.

21. The kit of claim 10, wherein barriers are placed between antigens to prevent cross-contamination.

22. The standardized assay kit of claim 1, wherein the assay is performed by hand.

23. The standardized assay kit of claim 1, wherein the assay is performed by an automated system.

22. A machine for automation of performance of a standardized stool based assay for the detection of anti-food antibodies comprising:
a holder for receiving a stool sample;
a centrifuge for the separation of solids from supernatant in a sample;
a second holder for receiving a test apparatus;
a fluid handling device for loading the sample supernatant onto the test apparatus;
a humid chamber for incubation of the test apparatus;
a detector to detect formation of a precipitate; and
a recorder to record detection of a precipitate.
